(19) United States
(12) Patent Application Publication
(10) Pub. No.: US 2024/0239654 A1
(43) Pub. Date: Jul. 18, 2024

(54) Production and Use of Liquid Fuel as a Hydrogen and/or Syngas Carrier

(71) Applicant: Infinium Technology, LLC, Sacramento, CA (US)

(72) Inventors: Robert SCHUETZLE, Sacramento, CA (US); Dennis SCHUETZLE, Grass Valley, CA (US); Anja Rumplecker GALLOWAY, San Rafael, CA (US); James BUCHER, Boston, MA (US); Harold WRIGHT, Brookfield, WI (US)

(21) Appl. No.: 18/445,228

(22) Filed: Jun. 5, 2023

Related U.S. Application Data
(62) Division of application No. 17/300,538, filed on Aug. 5, 2021.

(51) Int. Cl. C01B 3/32; B01J 8/02; B01J 8/06; B01J 23/50; B01J 23/63; B01J 23/652; B01J 23/80; B01J 35/61; C01B 32/50; C07C 29/151; C25B 1/04

(52) U.S. Cl. CPC C01B 3/326; B01J 8/0242; B01J 8/06; B01J 23/50; B01J 23/63; B01J 23/6525; B01J 23/80; B01J 35/613; B01J 35/615; C01B 32/50; C07C 29/1518; C25B 1/04; B01J 2208/00106; C01B 2203/0233; C01B 2203/0283; C01B 2203/107; C01B 2203/1223; C01B 2203/1229; C01B 2203/148; C01B 2203/1614; C01B 2203/1628

(57) ABSTRACT

The present invention is generally directed to the efficient production of low-carbon methanol, ethanol or mixtures of methanol and ethanol from captured CO₂ and renewable H₂ at a generation site. The H₂ is generated from water using an electrolyzer powered by renewable electricity, or from any other means of low-carbon H₂ production. An improved catalyst and process is described that efficiently converts H₂ and CO₂ mixture to syngas in a one-step process, and alcohols, such as methanol and ethanol, are produced from the syngas in a second step. The liquid methanol and ethanol, which are excellent H₂ carriers, are transported to a production site, where another improved catalyst and process efficiently converts them to syngas. The syngas can then be used at the production site for the synthesis of low carbon fuels and chemicals, or to produce purified low carbon H₂. The low carbon H₂ can be used at the production site for the synthesis of low-carbon chemical products or compressed for transportation use.

### CROSS REFERENCE TO RELATED APPLICATION

This application is a division of U.S. patent application Ser. No. 17/300,538, filed Aug. 5, 2021, which is incorporated by reference herein in its entirety.

### FIELD OF THE INVENTION

The field of this invention is the production of low-carbon methanol, ethanol, or other liquids from low carbon H₂ and CO₂ at distributed generation sites, and the transport of these low-carbon liquids to chemical and/or fuel production sites, or other locations where low carbon hydrogen or syngas is needed. Improved processes are described for the efficient production of syngas from the low carbon H₂ and CO₂. The syngas is used for the catalytic production of the alcohols or other liquids which are transported to one or more production sites where low carbon hydrogen or syngas is needed. H₂ is produced from the liquids at the production sites by catalytic steam reforming or other reforming or partial oxidation processes. Improved catalysts and processes are described for the efficient production of syngas from the liquids. This syngas is used to produce a variety of low-carbon fuels and chemicals. H₂ produced from the liquids may be used in applications including fuel cell vehicles, hydrocracking, green diesel production, ammonia production, chemicals production, and other applications that require low carbon, zero carbon, or negative carbon hydrogen. In some applications, the CO₂ produced from the production of H₂ from the liquid H₂ carrier is sequestered whereby the resulting H₂ produced at the production site is carbon negative H₂.

### BACKGROUND OF THE INVENTION

CO₂ can be generated and captured by several processes generally involving the combustion of fuels, the oxidation of chemicals, gasification processes, petroleum refining, natural gas power production, etc. CO₂ can also be produced through other industrial processes including ethanol production and other biological processes. In addition, emerging approaches to capturing CO₂ from air (called Direct Air Capture—DAC) allow for CO₂ capture from any location globally without being tied to an industrial source (Artz et al, 2018).

Since there are very few locations where suitable geological formations are available to sequester the CO₂, it is much more suitable to produce fuels and chemical products from the CO₂. Furthermore, CO₂ is a valuable feedstock to produce low-carbon fuels and chemicals.

Low carbon electricity can be used to produce H₂ using electrolysis (Equation 1). The low-carbon electricity can be generated from renewable power sources such as wind, solar, nuclear, hydroelectric, geothermal, biomass, biogas, or other ways to generate low carbon power.

$${H}_{2} O = {H}_{2} + \frac{1}{2} {O}_{2} \qquad Eq . 1$$

The electrolysis reaction uses the low carbon electricity to split water into H₂ and O₂. Electrolyzers consist of an anode and a cathode separated by an electrolyte. Electrolysis for H₂ production has been a working technology for many years. However, with decreases in the cost of renewable and low carbon electricity, interest in electrolysis has been increasing (Yan et al, 2019).

There are other forms of low carbon H₂ including blue H₂ (production of H₂ from natural gas or renewable gas reforming) that employs carbon capture and storage, turquoise H₂ (pyrolysis of natural gas/methane to produce H₂ and carbon black or other products that sequester the carbon), and other methods of production of low-carbon H₂.

Because H₂ is a useful low carbon fuel source, there has been an increased interest in moving H₂ from the generation site to the location of use. To date, H₂ has be transported primarily as liquid H₂ or compressed H₂ (Schwartz, 2011).

More recently Liquid Organic H₂ Carriers (LOHC) have been developed that hydrogenate the carrier molecule with H₂ resulting in a liquid organic compound that can be easily transported (Hurskainen et al, 2020).

The LOHC hydrogenation is an exothermic reaction (releases heat) and is carried out at elevated pressures (approx. 30-100 bar) at temperatures of approximately 150-250° C. in the presence of a catalyst. The corresponding liquid compound is thereby formed, which can be stored or transported under ambient conditions. The H₂ rich form of the LOHC is dehydrogenated, with the H₂ being released again from the LOHC. This reaction is endothermic (heat input required) which typically takes place at elevated temperatures (250-320° C.) in the presence of a catalyst. Before the H₂ can be used, it usually must be purified. The LOHC must usually be returned to the H₂ generation location to complete the cycle (Preuster et al, 2017).

There are also some drawbacks to LOHCs that might limit their economic feasibility in some cases. One distinct feature is the high reaction enthalpies, meaning that a significant amount of heat is required to release the H₂ from the LOHC. Considering the inevitable heat transfer losses, an approximate 25-30% of the released H₂ needs to be combusted if the heat is provided by H₂. Furthermore, as the required temperature level is quite high, it is not possible to use low-value waste-heat sources in most cases. One drawback is also that dehydrogenation must be carried out at close to atmospheric pressure, while hydrogenation in most cases requires some additional pressurizing. Where high-pressure H₂ is required by the user, such as bottle filling stations or mobility applications which leads to a high energy demand for compression. Compression of H₂ requires energy and adds capital costs.

Furthermore, the depleted LOHC must be delivered back to the H₂ generation source. This complicates delivery chains if the same truck needs to deliver H₂ to multiple locations in one trip. Lastly, the LOHC concept requires hydrogenation and dehydrogenation reactors, which increase H₂ delivery costs.

Low molecular weight alcohols (e.g., methanol, ethanol, and propanol) and low molecular weight acids (e.g., formic, and acetic acid)—are theoretically excellent LOHCs. In addition, naphtha, diesel, kerosene or other light hydrocarbons may also serve as excellent hydrogen carriers. H₂ can potentially be produced from these low-molecular weight alcohols and acids and hydrocarbons (Table 1).

TABLE 1
Potential Low Molecular Weight Liquid LOHC's

| | Mole % H₂ in | Relative Enthalpy of |
|---|---|---|
| Potential LOHC | Compound | Dehydrogenation to H₂ |
| Methanol (CH₃OH) | 6.25 | 1.00 |
| Ethanol (CH₃CH₂OH) | 6.50 | 1.17 |
| Propanol (CH₃CH₂CH₂OH) | 6.66 | 1.58 |
| Formic Acid (CH₂O₂) | 2.08 | 2.12 |
| Acetic Acid (CH₃CH₂O₂) | 3.33 | 2.18 |
| Naphtha (C₆-C₁₁) | 7.90 | 1.03 |

Methanol, ethanol, and propanol have about the same abundance of H₂, and the energy required to produce H₂ from methanol and ethanol is about the same, but the energy required to produce H₂ from propanol is 1.58 times greater than that of methanol. In addition, there is a greater probability of producing side products from ethanol and propanol than from the methanol. Formic acid and acetic acid are also potential LOHC's but the abundance of H₂ in these carriers is much lower than that of the alcohols.

The energy required to produce H₂ from naphtha, diesel, kerosene or a combination of some or all of these fractions (sometimes called e-crude) is about the same as that for methanol, but side products may be produced and therefore the yield of H₂ is lower.

Methanol can be produced from syngas according to Equation 2. Currently, methanol is primarily produced from natural gas and coal. The fossil fuels are first steam reformed to syngas followed by the production of the syngas to methanol (NETL, 2021). The problem is that the methanol produced from fossil fuels is not a low-carbon product.

$$2 {H}_{2} + C O = C {H}_{3} O H \qquad Eq . 2$$

Syngas can potentially be commercially produced from the catalytic conversion of low-carbon H₂ and captured CO₂ mixtures. This catalytic process is called the Reverse Water-Gas Shift (RWGS) reaction or CO₂ Hydrogenation (Equation 3) (Daza et al, 2016; Vogt et al, 2019; Chen et al, 2020).

$$C {O}_{2} + {H}_{2} = C O + {H}_{2} O \qquad Eq . 3$$

This reaction is endothermic at room temperature and requires heat to proceed. Elevated temperatures and efficient catalysts are required for significant CO₂ conversion to CO with minimal or no coking (carbon formation) or degradation in catalyst performance with time.

In addition to the production of alcohols, this low-carbon syngas is an excellent feedstock for producing a wide range of other chemical products, including liquid and gaseous hydrocarbon fuels, acetic acid, dimethyl ether, and many other chemical products (Olah et al, 2009; Centi et al, 2009; Jiang et al, 2010; Fischer et al, 2016; Li et al, 2019; NAS, 2019).

### SUMMARY OF THE INVENTION

H₂ is available from the electrolysis of water from low-carbon electricity or from other sources of low-carbon H₂ generation at the generation site. CO₂ is available from a nearby carbon capture facility, from direct air capture, from a CO₂ pipeline, or from other sources. Geological or other conditions at the generation site usually do not allow for the nearby sequestration of the CO₂.

At or near the generation site, captured CO₂ and low-carbon H₂ are processed in an improved two-step process to produce the liquid carrier. The liquid carrier can be any chemical or mixture of chemicals that is a liquid at ambient temperature and ambient pressure. For example, the liquid carrier may be a hydrocarbon or a mixture of hydrocarbons or the liquid carrier can be an alcohol such as methanol or ethanol, an acid, a chemical intermediary or a mixed alcohol or any other type of liquid carrier containing H₂ and carbon.

The carrier is produced from the captured CO₂ and renewable H₂ that are available at the generation site. As described earlier, methanol or ethanol are potentially excellent H₂ carriers.

Methanol is commercially produced from the catalytic reaction of H₂ and CO in a fixed-bed tubular reactor operating at about 50 bar and 275° C. over a Cu—ZnO-based catalyst (Equation 4). The products are methanol (97%); ethanol (2%); methane (1%) and acetic acid (1%) (Hurley, Schuetzle et al, 2010).

$$2 {H}_{2} + C O = C {H}_{3} OH [ Δ H = - 91 kJ / mol ( 298 °K ) ] \qquad Eq . 4$$

An alternative method is to produce methanol from CO₂ and H₂ according to Equation 5.

$$C {O}_{2} + 3 {H}_{2} = C {H}_{3} O H ( 1 ) + {H}_{2} O ( g ) [ Δ H = - 87 kJ / mol ( 298 °K ) ] \qquad Eq . 5$$

Ethanol can also be produced from the exothermic reaction of H₂ and CO at the generation site in a fixed-bed tubular reactor operating at about 50 bar and 275° C. over three catalysts in tandem reactors [#1: Cu—Zn-Alkali; #2: Rh—Y-Alkali; and #3 (Mo—Pd)]. The products are ethanol (72%); methanol (6%); methane (20%) and acetic acid (2%). (Hurley, Schuetzle et al, 2010) (Equation 6).

$$4 {H}_{2} + 2 CO = {CH}_{3} C {H}_{2} O H ( 1 ) + {H}_{2} O ( g ) [ Δ H = - 298 kJ / mo l ( 298 °K ) ] \qquad Eq . 6$$

In addition, as summarized in the next section, no commercial catalysts have been successfully developed to date for the dehydrogenation of methanol or ethanol to produce syngas or H₂.

Catalytic Reforming of Methanol—Since methanol is a liquid, it can be easily transported to a second site, the conversion site. If the objective is only to utilize H₂ at the conversion site, then the H₂ may be produced by catalytic steam reforming of the methanol which is an endothermic process according to Equation 7 (Palo et al, 2007; Iulianelli et al, 2014; Dalena et al, 2018).

$$C {H}_{3} O H ( g ) + {H}_{2} O ( g ) = C {O}_{2} + 3 {H}_{2} Δ H = + 50 kJ / mol ( 298 °K ) \qquad Eq . 7$$

The catalysts used for reforming govern the methanol conversion rate and the ratio of products (CO₂, H₂ and CO). Group VIII-XIII metals (primarily Cu, Pd and Zn) with different promoters have been widely utilized for methanol steam reforming since they produce a higher H₂ yield. A semi-empirical model of the kinetics of the catalytic steam reforming of methanol over CuO/ZnO/Al₂O₃ catalyst has been developed (Amphlett et al, 1994).

A mixture of water and methanol with a molar concentration ratio of 1.0-1.5/1.0 is pressurized to approximately 20 bars, vaporized and heated to a temperature of 250-360° C. The H₂ that is created is separated using pressure swing adsorption or a H₂-permeable membrane (Dalena et al, 2018; Ranjekar and Yadav, 2021).

With either design, not all the H₂ is removed from the product gases (raffinate). Since the remaining gas mixture still contains some chemical energy, it is often mixed with air and burned to provide additional heat for the endothermic reforming reaction.

CO₂ is emitted (Equation 7) which decreases the life-cycle greenhouse gas value of the H₂ produced. If the CO₂ can be sequestered or commercially utilized at the conversion site, then the life-cycle greenhouse gas impact can be reduced.

Some of the H₂ can be used as fuel for electricity generation or to produce fuels and chemicals. The H₂ may also be used for hydrotreating applications in refineries, green diesel facilities, fuel cell vehicles, hydrocracking, ammonia production, chemicals production, and other applications that require low carbon, zero carbon, or negative carbon hydrogen.

It is preferable to convert the methanol to CO and H₂ since CO₂ is not produced. The H₂ and CO can be separated or since the ratio of H₂/CO has the ideal stoichiometry (1.5-2.5/1.0) it can be used to produce low-carbon fuels and chemicals (Schuetzle et al, 2010 and 2016). The production of CO and H₂ can be catalytically converted from methanol according to the endothermic reaction given by Equation 8. The conversion of methanol to H₂ has been typically referred to as methanol dehydrogenation.

$$C {H}_{3} O H ( g ) = C O + 2 {H}_{2} Δ H = + 91 kJ / mol ( 298 °K ) \qquad Eq . 8$$

The H₂ and CO produced from the low-carbon methanol are low-carbon products. Therefore, if the H₂ and CO are separated and the CO used as an energy resource for the production processes, then the CO₂ is a low-carbon emission.

A few potential catalysts have been synthesized and tested for the reforming of methanol to syngas. It has been found that transition metal catalysts usually suffer from poor coke resistance during the dehydration of hydrocarbons in gas-solid heterogenous systems, including methanol dehydration.

Han et al (2020) synthesized and tested a catalyst comprised of atomically dispersed Ni sites in nitrogen-doped carbon sheets. They synthesized two types of catalysts. The composition of catalyst #1 was 18.3 wt. % Ni on nitrogen-doped carbon and catalyst #2 was produced by acid etching of catalyst #1 which reduced the Ni to 9.3 wt. %. These catalysts were tested for the conversion of methanol to CO and H₂ at 15 psig, a space velocity of 16,000 h⁻¹, and a temperature of 350° C. Catalyst #1 and catalyst #2 had losses in activity of 44% and 2%, respectively after 24 hours. In conclusion, these carbon-based catalysts are difficult to synthesize, they are very fragile, and their reductions in performance with time are not acceptable.

Carraro et al (2018) synthesized and tested a 5 wt. % Pd on CeO₂ catalysts for the conversion of methanol to syngas at 300° C. The methanol conversion efficiency was 95% for the first five hours of conversion but dropped to about 85% after fifteen hours. Surface analysis of the catalyst demonstrated that the drop in performance was the result of carbon formation on the surface of the catalyst.

Matsumurakoji et al (2000) synthesized a 10 wt. % Ni on silica catalyst and studied its performance for the decomposition of methanol to CO and H₂ at 350° C. They found that carbon deposited on the catalyst after 30 hours which significantly reduced the catalyst efficiency.

### Catalytic Reforming of Methanol and Ethanol

An improved catalyst and process has been developed and is described herein that contains no or low precious metals and which consists of low-cost, readily abundant metals.

One preferred catalyst is a Pd—Ag catalyst. Another preferred catalyst is a nickel solid solution catalyst. Another catalyst is a metal alumina spinel impregnated with one or more Group I and Group 2 elements.

This catalyst is comprised of a metal alumina spinel substrate or any other alumina substrate that has a surface area greater than about 50 m²/g that has been impregnated with one or more of the following (Cu, Mg, Ni and Zn) elements at a combined concentration of up to 15 parts-by-weight and up to 5 wt. % of La or Ce, and wherein the metal alumina spinel is selected from a group consisting of magnesium aluminate, calcium aluminate, strontium aluminate, potassium aluminate and sodium aluminate. The catalyst consists of one or more substitutional solid solutions on the metal impregnated metal-alumina spinel.

This improved catalyst converts methanol or ethanol to syngas with a per pass efficiency of greater than 60% and 45%, respectively, at 100-450 psig, 400-550° F. and a space velocity of 5,000-25,000 hr⁻¹. The syngas produced from the alcohols have an H₂/CO ratio of 1.8-2.2/1.0.

Since methanol and ethanol are acidic in nature, they react readily with basic surfaces of this catalyst that have abundant hydroxy (M-OH) groups to form HCOO-M and H₂ as shown in Equation 9 for methanol.

$$C {H}_{3} O H + M - O H = H C O O - M + 2 {H}_{2} \qquad Eq . 9$$

CO is then quickly released at the catalyst operating temperatures from the complex as shown by Equation 10.

$$HCOOH - M = M - O H + CO \qquad Eq . 10$$

Therefore, the combination of processes from Equations 9 and 10 produce syngas with an H₂/CO ratio of 2.0/1.0. In addition, the original catalyst surface M-OH is regenerated.

As described, formic acid (HCOOH) is also a possible H₂ carrier. It also reacts readily with the catalyst basic surface (M) that have abundant hydroxy (OH) groups to form formates (HCOO-M) as shown in Equation 11.

$$HCOOH + M - O H = H C O O - M + {H}_{2} \qquad Eq . 11$$

The CO is quickly released from the complex at the catalyst operating temperature according to Eq. 10. Therefore, the combination of processes from Equations 10 and 11 produce syngas with an H₂/CO ratio of 1.0/1.0.

Ethanol reacts with the basic catalyst surface in the same manner as methanol and it produces syngas with an H₂/CO ratio of 2.0/1.0. As shown previously in Table 1, methanol and ethanol contain 6.25 and 6.50 mole % H₂, respectively. However, the catalytic dehydrogenation of ethanol requires a higher catalyst operating temperature than that of methanol.

Methanol is a better H₂ carrier than formic acid since it produces twice as much H₂ for every carrier molecule. An advantage is that liquid H₂ carriers such as methanol and ethanol are completely utilized at the generation site and the solid materials, typically used in LOHC, do not need to be returned to the generation site.

The transport of H₂ carriers such as methanol, ethanol, naphtha, diesel and other liquid carriers is well known since they can use existing transportation infrastructure including truck, rail, barge, and other forms of transporting liquid fuels that are in widespread use today. The cost of the liquid H₂ carrier transportation is significantly lower than the cost of transporting compressed H₂ in tube trailers or liquid H₂ in specialized trucks equipped to handle liquid H₂.

### BRIEF DESCRIPTION OF THE DRAWINGS

FIG. 1 shows the overall process showing connection of the generation site (100) and the conversion site (200).

FIG. 2 shows some of the possible processes that are accomplished at the generation site (100). Block 101 is the electrolyzer block or low carbon H₂ production block. Block 102 is the Reverse Water-Gas Shift (RWGS) reactor system which converts CO₂ and H₂ into syngas (carbon monoxide and H₂). Block 103 is the conversion block that produces the liquid H₂ carrier (e.g., methanol) (stream 4).

FIG. 3 shows some of the possible process that are accomplished at the conversion site (200). Block 201 is the system for conversion of the liquid H₂ carrier into H₂ and CO₂ or H₂ and CO. Block 202 is the electricity generation block.

### DETAILED DESCRIPTION OF THE INVENTION

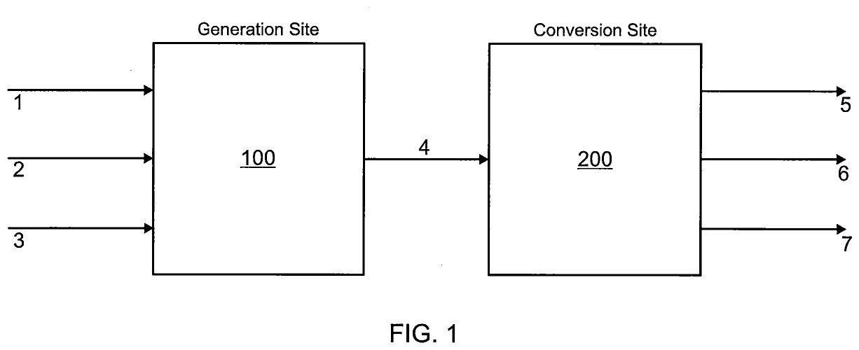

In FIG. 1, the generation site is shown as block 100. The generation site has at least three input streams. Stream 1 is an input stream that comprises low carbon electricity which can be generated by renewable sources such as wind and solar, from nuclear power plants, from hydroelectric, or from geothermal power plants. In one embodiment, the low carbon electricity can come from battery storage cells where the batteries were charged with intermittent electricity from wind farms or photovoltaic (PV) arrays. Block 100 may also represent other processes producing low carbon H₂, such as blue H₂, turquoise H₂, or other forms of low carbon H₂.

Stream 2 is a stream comprising high-purity water. The water can be from any source that meets the water quality specifications required for different electrolysis systems or may be recycled water from the production system.

Stream 3 is a stream comprising CO₂. CO₂ is available from a nearby carbon capture facility, from a carbon dioxide pipeline, or captured from ambient air. CO₂ can be generated and be captured by several processes generally involving the combustion of fuels, the oxidation of chemicals, gasification processes, petroleum refining, cement production, etc. For example, industrial manufacturing plants that produce ammonia for fertilizer produce large amounts of CO₂. Ethanol plants that convert corn or wheat into ethanol produce large amounts of CO₂ via fermentation. Power plants that generate electricity from various carbonaceous resources produce large amounts of CO₂. Municipal sewage treatment systems using aerobic and anaerobic digestion of sludge produce large amounts of CO₂. All these sources of CO₂ can be used in the current invention.

The CO₂ in stream 3 (FIG. 1) can be captured via standard means including using amine solvents. Utilization or conversion of CO₂, as described herein, typically involves separating and purifying the CO₂ from a gaseous stream where the CO₂ is not the major component (e.g., exhaust flue gas). Typically, an alkylamine is used to remove the CO₂ from the gas stream. Alkylamines used in the process include monoethanolamine, diethanolamine, methyldiethanolamine, diisopropylamine, aminoethoxyethanol, or combinations thereof. Metal Organic Framework (MOF) materials have also been used as a means of separating CO₂ from a dilute stream using chemisorption or physisorption. Other methods to collect concentrated CO₂ include chemical looping combustion where a circulating metal oxide material captures the CO₂ produced during the combustion process. CO₂ can also be captured from the atmosphere in what is called direct air capture (DAC). The processes for the capture of CO₂ often involve regeneration of the capture materials. Alkylamines are regenerated by being heated, typically by low pressure steam.

In FIG. 1, block 100 is the overall multi-step process for the generation of the liquid carrier. This is the generation site of the invention. At least one of the products of block 100 is shown as stream 4 which is a stream that comprises the liquid carrier.

At least a portion of the liquid carrier, stream 4, is transported to another location called the conversion site of the invention shown in FIG. 1 as block 200. The liquid carrier can be moved or transported by many different means to the conversion site. These means include by rail, truck, barge or boat, pipeline, or other methods for transporting liquid fuels, liquid hydrocarbons, or other liquid products. At the conversion site, 200, the liquid carrier, 4, is converted into several potential products.

FIG. 1 shows a stream comprising low carbon electricity denoted as stream 5 leaving the 200 block. FIG. 1 also shows a stream comprising syngas shown as stream 7 leaving the conversion site, block 200. A stream comprising H₂, shown as stream 6, is one of the products of the conversion site, block 200. Either the H₂ or the syngas may be the primary product of interest.

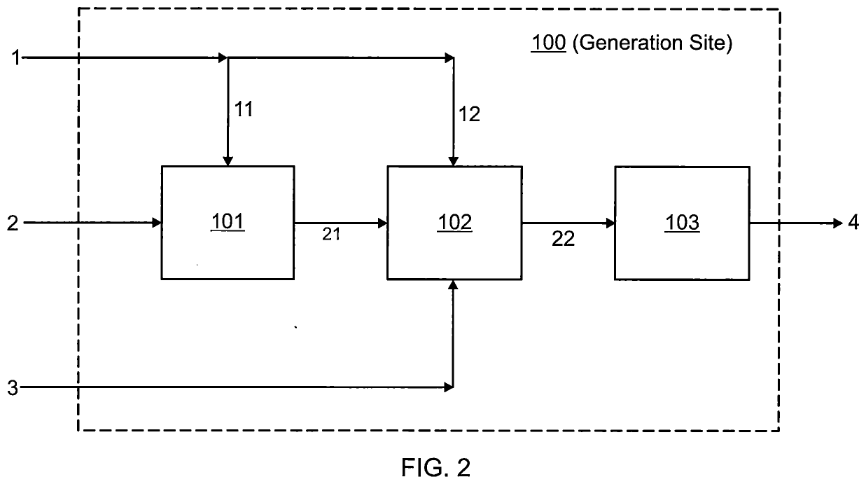

FIG. 2 shows some of the possible processes that can be accomplished at the generation site (100). Block 100 is shown as the dashed box around boxes 101, 102, and 103. Block 101 is the low carbon H₂ generation block using electrolysis. Low carbon electricity, stream 1, and a stream comprising purified water, stream 2, are the inputs to the low carbon H₂ generation block, 101. The low carbon H₂ generation block may utilize an electrolyzer, 101, which comprises an anode and a cathode separated by an electrolyte. Different electrolyzers function in slightly different ways. Different electrolyzer designs can be used in the invention including alkaline electrolysis, membrane electrolysis, and high temperature electrolysis. Different electrolytes can be used including liquids KOH and NaOH, and with or without activating compounds. Activating compounds can be added to the electrolyte to improve the stability of the electrolyte. Most ionic activators for the H₂ evolution reaction are composed of an ethylenediamine-based metal chloride complex and Na₂MoO₄ or Na₂ WO₄. Different electro-catalysts can be used on the electrodes including many different combinations of metals and oxides like Raney-Nickel-Aluminum, which can be enhanced by adding cobalt or molybdenum to the alloy.

Several combinations of transition metals, such as Pt₂Mo, Hf₂Fe, and TiPt, have been used as cathode materials and have shown significantly higher electrocatalytic activity than state-of-the-art electrodes.

Water at the anode combines with electrons from the external circuit to form oxygen gas, positively charged H₂ ions, and electrons. The H₂ ions pass through the membrane and combine with the electrons from the external circuit at the cathode to form H₂ gas. In this way, both H₂ and O₂ are produced in the electrolyzer. In one embodiment of the invention, multiple electrolyzers are operated in parallel.

The electrolyzer produces at least two product streams, a H₂ stream 21 (FIG. 2), and an oxygen stream, not shown in FIG. 2.

Block 102 in FIG. 2 is the RWGS reactor system. In one embodiment of the invention, the multistep process to produce the liquid carrier at the generation site, 100, involves the RWGS reactor where CO₂ is first converted to CO. The RWGS reactor is used to convert the carbon dioxide from stream 3 and electrolyzer H₂, stream 21, into a RWGS reactor product by the endothermic reaction previously denoted by Equation 3.

The RWGS reactor feed streams, stream 21 and stream 3, are blended in Block 102. The ratio of H₂/CO₂ in the RWGS feed stream is between 2.0 mol/mol to 5.0 mol/mol or more preferably between 3.0 mol/mol and 4.0 mol/mol. The mixed RWGS reactor feedstock must be heated to RWGS operating conditions. In one embodiment, the RWGS feed stream is heated to reaction temperature of greater than 1450° F. (e.g., between 1,450 and 1,800° F.), or preferably greater than 1,550° F. (e.g., between 1,550 and 1,750° F.) using a RWGS feed heater. In one embodiment, the RWGS feed heater is a fired heater that uses the combustion of H₂ taken from stream 21 as the fuel gas that combusts with air to produce water and heat. This heat is used to raise the temperature of the RWGS feed stream.

In another embodiment, low carbon electricity is used in an electrical heater to raise the RWGS feed temperature. The heater is electrically heated and raises the temperature of the feed gas through indirect heat exchange to greater than 1,550° F. (e.g., between 1,550 and 1,750° F.).

The heated RWGS reactor feed gas is supplied to a main RWGS reactor. The main reactor vessel is adiabatic or nearly adiabatic and is designed to minimize heat loss. No heat is added to the main reactor vessel.

The product stream leaving the main RWGS reactor vessel (102) are comprised of CO, unreacted H₂, unreacted CO₂, and H₂O. Additionally, the product stream may also comprise a small amount of methane (CH₄) that was produced as a side reaction.

The product stream is shown in FIG. 2 as stream 22. Stream 22 can be used in a variety of ways at this point in the process. The product gas can be cooled and compressed and used in downstream process to produce fuels and chemicals (Tan et al, 2018) (Schuetzle et al patents, 2010-2019). The product stream can also be cooled, compressed, and sent back to the preheater and fed back to the main reactor vessel. The product stream can also be reheated in a second electric preheater and sent to a second reactor vessel where additional conversion of CO₂ to CO can occur.

In other embodiments, where CO is not used in the conversion block, 103, block 102 does not act as a RWGS reactor system but only acts only to mix and heat the feeds for block 103 listed as stream 22 in FIG. 2. In these embodiments, the conversion block, 103, involves the direct hydrogenation of CO₂ to produce a product stream 4.

Block 103 of FIG. 2 is the conversion block that produces a liquid product (stream 4). In one embodiment, the conversion block comprises a Liquid Fuel Production (LFP) reactor system where the carbon monoxide and the H₂ that are in stream 22 are converted directly to a mixture of liquid hydrocarbons, alcohols or other liquid hydrogen carriers.

At least a portion of the RWGS product gas is used as the Liquid Fuel Production (LFP) reactor feed. Also, because the operating pressure of the RWGS reactor may be lower than that of the LFP operating pressure, the produced syngas may require compression to the LFP inlet pressure. The LFP is also known as the hydrocarbon synthesis step. The LFP reactor converts CO and H₂ into C₅-C₂₄ hydrocarbons that can be used as liquid fuels and chemicals and, in this case, produces the liquid carrier, stream 4. Ideally the H₂ to CO ratio in the feed to the LFP reaction is between 1.9 and 2.2 mol/mol but it may be below 1.9 or above 2.2 as necessary to modify the composition of the liquid stream. The LFP reactor is a multi-tubular fixed bed reactor system. The LFP reactor tube profile can be round, oval, flattened, twisted, or other variations. The LFP reactors are generally vertically oriented with LFP reactor feed entering at the top of the LFP reactor. However, horizontal reactor orientation is possible in some circumstances and setting the reactor at an angle may also be advantageous in some circumstances where there are height limitations. Most of the length of the LFP reactor tube is filled with LFP catalyst. The LFP catalyst may also be blended with diluent such as silica or alumina to aid in the distribution of the LFP reactor feed into and through the LFP reactor tube.

The LFP reactor in one embodiment is operated at pressures between 150 to 450 psig. The reactor is operated over a temperature range from 350° F. to 460° F. and more typically at around 410° F. The LFP reaction is exothermic in which the temperature of the reactor is maintained inside the LFP reactor tubes by the reactor tube bundle being placed into a heat exchanger where boiling water is present on the outside of the LFP reactor tubes. The boiler water temperature is at a lower temperature than the LFP reaction temperature so that heat flows from the LFP reactor tube to the lower temperature water. The shell water temperature is maintained by controlling the pressure of the produced steam. The steam is generally saturated steam. In alternate embodiments, the catalytic LFP reactor can be a slurry reactor, microchannel reactor, fluidized bed reactor, or other reactor types known in the art.

The CO conversion in the LFP reactor is maintained at between 30 to 80 mole % CO conversion per pass. Unconverted gas can be recycled for extra conversion or sent downstream to an additional LFP reactor. Multiple LFP reactors may also be used in series or in parallel.

In one embodiment, a series of fractionators are used to create a high cetane diesel fuel with an adjustable flash point, and a stabilized naphtha (potentially a gasoline blend stock or chemical feedstock) or a blended e-crude. The high cetane diesel fuel can be used as a liquid carrier (FIG. 2, Stream 4). The unfractionated liquid hydrocarbon (e-crude) from the LFP reactor can also be used as the liquid carrier.

In another embodiment, an alcohol synthesis reactor is used as the conversion device of Block 103 (FIG. 2). In this embodiment, the synthesis gas (H₂ and CO) from the RWGS reactor product is converted to a product comprising an alcohol. Methanol is a common alcohol that can be produced from syngas that in some embodiments can be used as a liquid H₂ carrier.

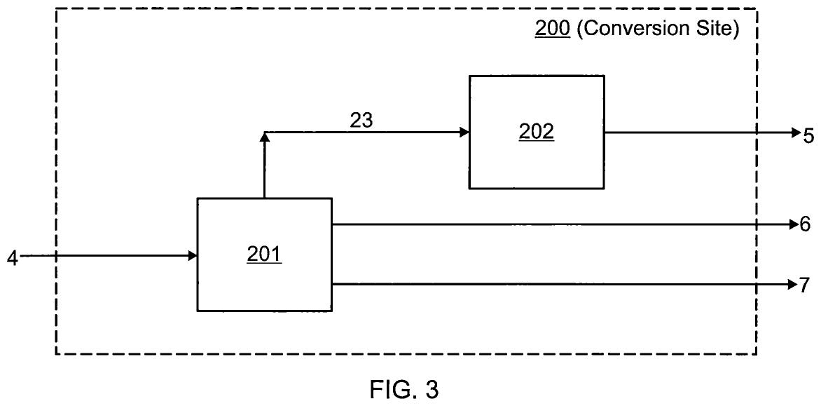

FIG. 3 shows some of the processes that may be performed at the conversion site. Block 201 is the CO₂ separation block if the conversion process produces CO₂. Block 202 is the electricity generation block.

The CO₂ separation block (Block 201) comprises a means in which, at a minimum, a stream comprising CO₂ (Stream 7), and a means to produce electricity, shown as stream 23, are produced from the chemical conversion of the liquid carrier, stream 4. Optionally, a stream comprising H₂ shown as stream 6 can be produced in Block 201. Block 201 may comprise multiple steps or processes for the conversion.

The CO₂ separation block can be accomplished by several means that include steam reforming. In one embodiment, the liquid carrier comprises a system where the liquid carrier, 4, is reacted with steam to produce a product mixture of H₂ and CO₂ that can be separated into streams 6 and 7, respectively. In another embodiment, the liquid carrier can be steam reformed to produce a mixture of H₂ and CO as shown by Equation 7. The H₂ in the steam reformer product can be separated to become stream 6.

A mixture of water and methanol with a molar concentration ratio (water/methanol) of 1.0-1.5 is pressurized to approximately 300 psig, vaporized, and heated to a temperature of 250-360° C. The H₂ that is created is separated using pressure swing adsorption (PSA), an H₂-permeable membrane, or a palladium alloy. There are two basic methods of conducting this process.

The water-methanol mixture is introduced into a tube-shaped reactor where it contacts the catalyst. H₂ is then separated from the other reactants and products in a later chamber, either by PSA or through use of a membrane where the majority of the H₂ passes through.

The other process features an integrated reaction chamber and separation membrane, a membrane reactor. The reaction chamber is made to contain high-temperature, H₂-permeable membranes that can be formed of refractory metals, palladium alloys, or a Pd/Ag-coated ceramic. The H₂ is thereby separated out of the reaction chamber as the reaction proceeds. This purifies the H₂ and, as the reaction continues, increases both the reaction rate and the amount of H₂ extracted.

With either design, not all the H₂ is removed from the product gases. Since the remaining gas mixture still contains a significant amount of chemical energy, it can be mixed with air and burned to provide heat for the endothermic reforming reaction.

In one embodiment, the steam reforming system comprises an adiabatic reactor with limited heat loss. The steam reformer feed is heated in a steam reformer heater. In one embodiment, the steam reformer heater is heated to temperature by low carbon electricity through an electrical heater. In another embodiment, the steam reformer heater is heated to temperature by the combustion of H₂ or a combination of H₂, CO, and unconverted vaporized liquid carrier. In another embodiment, the steam reformer is operated at nearly isothermal conditions and the steam reformer feed is fed through multiple tubes that are in a heater fire box. In another embodiment, the steam reforming is performed using waste heat from an industrial facility or co-location facility at the conversion site. The combustion of H₂ or a combination of H₂, CO, and unconverted vaporized liquid carrier provides the heat of reaction. In one embodiment, the steam reformer feed is heated by cross exchange with the steam reformer product and additional heat from the electricity generation block, block 202.

In another embodiment, the CO₂ conversion block, 201, comprises an oxy-combustion system in which the liquid carrier, stream 4, is reacted with nearly pure oxygen to produce a stream comprising CO₂ and H₂O. The oxy-combustor product stream can be separated such that a CO₂ rich stream, stream 7, is produced. The oxygen is produced by an air separation unit or is available by pipeline.

In another embodiment, the CO₂ conversion block, 201, comprises a partial oxidation system in which the liquid carrier, stream 4, is reacted with nearly pure O₂ at a rate to accomplish the partial oxidation to a partial oxidation product comprising H₂ and CO. The O₂ to C ratio is controlled to approximately 0.50 to 0.55 on a molar basis to allow the partial oxidation instead of full combustion. This partial oxidation stream can be separated by PSA or other means to produce a nearly pure stream comprising H₂, stream 6. The CO can be converted to CO₂ through water gas shift or through further oxidation such that a stream comprising CO₂ is produced, stream 7.

Both the oxy-combustion and the partial oxidation embodiments are exothermal under normal operation and require no additional external heat to heat the feed gas to full operating temperature, unlike the steam reforming embodiments.

The CO₂ conversion reactors often result with product streams that are at elevated temperature. In one embodiment, a heat recovery system can be used to reduce the product gas temperature. Steam is generated in the cooling of the gas. The steam so generated in this embodiment is stream 23 and provides the motive force for the generation of electricity in electricity generating block, 202. In other embodiments, the high temperature heat and some portion of the product gas that is conveyed from the CO₂ conversion reactors can be stream 23 that acts as a feed gas to a solid oxide fuel cell as one embodiment of the electricity generation block, 202.

The electricity generating block, 202, can be any number of electricity generation systems. These systems may include but are not limited to steam turbine systems, fuel cell systems, gas turbine systems, organic Rankine cycle systems, or Stirling engines systems.

### Example 1: Methanol as a H₂ and Carbon Dioxide Carrier

A stream comprising CO₂ is produced by an industrial process or captured from ambient air. This CO₂ stream is fed to a CO₂ capture facility. The CO₂ capture facility uses methyl diethanolamine (MDEA) in an absorber tower to capture the CO₂. Relatively pure CO₂ (FIG. 1, Stream 3) is regenerated from the MDEA by heating.

Low-carbon electricity from a wind farm, a solar farm, a nuclear power plant, or other low-carbon power sources is available at the site of the carbon capture facility. High-purity water is produced from locally available water. Low-carbon H₂ is produced from the purified water via electrolysis.

This reaction uses the low-carbon electricity to split the water into H₂ and O₂. The electrolyzer in this example is a PEM Electrolyzer, block 101 in FIG. 2. The electrolyzer produces two streams, H₂ (FIG. 2, Stream 2) and O₂ (stream not shown).

At the generation site, 100, the improved catalyst and catalytic reactor (FIG. 2, Block 102), is used to convert the captured CO₂ stream and renewable H₂ stream into a product stream 22. In this example, low-carbon electricity, stream 12, is used to supply the electricity to power an electrical heater that raises the RWGS feed stream to about 1650° F. In this example, the H₂ to CO₂ ratio is 3.4/1.0, the pressure is 300 psig, and the space velocity is 20,000 hr⁻¹. Example 1 provides the relationship between temperature and % CO₂ conversion to CO for the improved RWGS catalyst. The conversion of CO₂ is about 82% under these conditions.

The product stream, stream 22 (FIG. 2), is compressed to about 50 atmospheres to produce a methanol feed stream. The conversion block, 103, in this example is a methanol reactor system. The H₂ and CO are converted to methanol using a Cu—ZnO based catalyst in a fixed bed reactor operated at about 275° C.

At least a portion of the methanol produced in the syngas-to-methanol reactor is the liquid carrier, stream 4, and is transported to a second site by rail, truck or by other suitable means, 200 (FIG. 3).

At the conversion site, the transported methanol is converted to a stream of H₂ and CO₂ by a steam reforming process, 201 (FIG. 3). The transported methanol is first stored in a storage tank. The methanol is pumped from the storage tank, mixed with water, and heated to about 275° C. by indirect heat exchange (where at least one of the reformer heat exchangers is a reformer feed and product cross-exchange heat exchanger in this case). The steam to carbon ratio in the water-methanol mix is controlled to 1.5 on a molar basis. The heated methanol-water mixture is fed to a reformer reactor where the methanol is reacted by the steam reforming reaction shown by Equation 12 to produce CO₂ and H₂.

$$C {H}_{3} O H ( g ) + {H}_{2} O ( g ) = C {O}_{2} + 3 {H}_{2} Δ H = + 50 kJ / mol ( 298 °K ) \qquad Eq . 12$$

In this example, the catalyst in the reformer is a Pd—Ag catalyst. In another embodiment, the catalyst is a nickel solid solution catalyst, and in yet another embodiment the catalyst is a metal alumina spinel impregnated with one or more Group I and Group 2 elements.

The reactor is an adiabatic fixed bed reactor with a pressure drop of less than 25 psig across the reactor and catalyst bed. Over 95% (i.e., between 95% and 100%) of the methanol in the reformer feed is converted to CO₂ and H₂.

Some water and carbon monoxide are also present in the methanol reformer product. The methanol reformer product is cooled via cross exchange with the reformer feed stream. The methanol reformer product in this example is further processed in a pressure swing adsorption (PSA) unit to recover the H₂. Before the PSA unit, most of the water in the reformer product stream is removed in a knock-out vessel. The knock-out vessel overheads become the feed to the PSA unit. The knock-out vessel bottoms which are predominantly water are recycled and used as a portion of the water that is blended with the methanol stream to produce the methanol reformer feed stream. The pressure swing adsorption unit comprises beds of solid adsorbent to separate impurities from the H₂-rich methanol reformer product stream. The higher-pressure H₂ in the reformer product and PSA feed stream is absorbed on the adsorbent. The adsorbent beds “swing” between impurity adsorbing and desorbing operations. This leads to a high-pressure PSA product stream that has a composition of over 95 volume % H₂ at a pressure of 176 psig. The low-pressure PSA product is called tail-gas and has a pressure of 20 psig, a molar composition of approximately 22% H₂, 71% CO₂, 5% CO, and 2% water. Overall, through the PSA Unit, 90% of the H₂ in the reformer product stream ends up in the high-pressure PSA product stream, stream 6.

The low-pressure PSA product stream still has H₂, CO, and a small volume of unconverted vaporized liquid carrier that can be used to produce electricity, stream 23.

### Example 2: Ethanol as a CO₂ Carrier

A stream comprising CO₂ is produced by an industrial process or captured from ambient air. This CO₂ stream is fed to a CO₂ capture facility. The CO₂ capture facility uses methyl diethanolamine (MDEA) in an absorber tower to capture the CO₂. Relatively pure CO₂ (FIG. 1, Stream 3) is regenerated from the MDEA by heating.

Low-carbon electricity from a wind farm, a solar farm, a nuclear power plant, or other low-carbon power sources is available at the site of the carbon capture facility. High-purity water is produced from locally available water. Low-carbon H₂ is produced from the purified water via electrolysis.

This reaction uses the low-carbon electricity to split the water into H₂ and O₂. The electrolyzer in this example is a PEM Electrolyzer, block 101 in FIG. 2. The electrolyzer produces two streams, H₂ (FIG. 21, Stream 2) and O₂ (stream not shown).

At the generation site, 100, the improved catalyst #1 and catalytic reactor (FIG. 2, Block 102), is used to convert the captured CO₂ stream and renewable H₂ stream into a product stream 22. In this example, low-carbon electricity, stream 12, is used to supply the electricity to power an electrical heater that raises the RWGS feed stream to about 1650° F. In this example, the H₂ to CO₂ ratio is 3.4/1.0, the pressure is 300 psig, and the space velocity is 20,000 hr⁻¹. Example 1 provides the relationship between temperature and % CO₂ conversion to CO for the improved catalyst #1. The conversion of CO₂ is about 82% under these conditions with less than 0.50% CH₄ selectivity.

The product stream, stream 22 (FIG. 2), is compressed to about 50 atmospheres to produce an ethanol reactor feed stream. The conversion block, 103, in this example is an ethanol reactor system. The H₂ and CO are converted to ethanol using three catalysts in tandem reactors [#1: Cu—Zn-Alkali; #2: Rh—Y-Alkali; and #3 (Mo—Pd)]. The products are ethanol (72%); methanol (6%); methane (20%) and acetic acid (2%). (Hurley, Schuetzle et al, 2010).

At least a portion of the ethanol produced in the syngas-to-ethanol reactor is the liquid carrier, stream 4 (FIG. 2), and is transported to a second site, 200 (FIG. 3). In this example, the liquid is transported by rail or truck to the conversion site, 200.

The transported ethanol is first stored in a storage tank. The ethanol is pumped from the storage tank at 200 psig, mixed with water, and heated to 260° C. by indirect heat exchange (where at least one of the reformer heat exchangers is a reformer feed and product cross-exchange heat exchanger in this case). The steam to carbon ratio in the water-ethanol mix is controlled to 1.5 on a molar basis. The heated ethanol-water mixture is fed to a catalytic reactor where the ethanol is converted to a stream of H₂ and CO₂, 201 (FIG. 3) as by the steam reforming process shown by Equation 13.

$$C {H}_{3} C {H}_{2} O H ( g ) + 3 {H}_{2} O ( g ) = 2 C {O}_{2} + 6 {H}_{2} Δ H = + Z kJ / mol ( 298 °K ) \qquad Eq . 13$$

In this example, the catalyst in the reformer is a Pd—Ag catalyst. In another embodiment, the catalyst is a nickel solid solution catalyst and in yet another embodiment the catalyst is a metal spinel impregnated with one or more Group I and Group 2 elements.

The reactor is an adiabatic fixed bed reactor with a pressure drop of 14 psi across the reactor and catalyst bed. Over 99% (i.e., between 99% and 100%) of the ethanol in the reformer feed is converted to H₂ and CO₂. Some H₂O and CO are also present in the ethanol reformer product. The ethanol reformer product is cooled via cross exchange with the reformer feed stream. The ethanol reformer product in this example is further processed in a pressure swing adsorption (PSA) unit to recover the H₂. Before the PSA unit, most of the H₂O in the reformer product stream is removed in a knock-out vessel. The knock-out vessel overheads become the feed to the PSA unit. The knock-out vessel bottoms which are predominantly water are recycled and used as a portion of the water that is blended with the methanol stream to produce the methanol reformer feed stream. The pressure swing adsorption unit comprises beds of solid adsorbent to separate impurities from the H₂ rich methanol reformer product stream. The higher-pressure H₂ in the reformer product and PSA feed stream is absorbed on the adsorbent. The adsorbent beds “swing” between impurity adsorbing and desorbing operations. This leads to a high-pressure PSA product stream that has a composition of over 99 volume % H₂ at a pressure of 176 psig. The low-pressure PSA product is called tail-gas and has a pressure of 20 psig, a composition by mole of about 22% H₂, 71% CO₂, 5% CO, and 2% H₂O. Overall, through the PSA Unit, 90% of the H₂ in the reformer product stream ends up in the high-pressure PSA product stream, stream 6.

The low-pressure PSA product stream still has H₂, CO, and a small amount of unconverted vaporized liquid carrier that can be used to produce electricity, stream 23.

### Various Processes and Catalysts

The present invention provides various processes and catalysts. In one aspect, the present invention provides a process “A” for utilizing captured carbon dioxide at a generation site. The process A involves: producing a hydrogen stream from water using an electrolyzer powered by low carbon electricity; utilizing a carbon dioxide stream from a carbon capture facility or a carbon dioxide pipeline; catalytically converting the hydrogen stream with the carbon dioxide stream to produce a low carbon syngas (e.g., H₂ and CO mixture); catalytically converting the low carbon syngas to a liquid, low carbon H₂ carrier; transporting at least a portion of the liquid (e.g., 10% to 100%) to a production site; catalytically converting the liquid low carbon H₂ carrier to H₂ or syngas.

The H₂ produced from the liquid H₂ carriers from process A can be used: as a fuel for vehicles; for the production of chemicals; for the production of power; for the production of green diesel; directly to produce low-carbon liquid fuels; directly to produce low-carbon chemicals; in the production of low carbon diesel, naphtha and jet fuel; for the production of low carbon, high-value chemical products; for the production of power.

The liquid hydrogen carrier produced in process A can be any suitable carrier, including: methanol, ethanol, propanol, a methanol/ethanol mixture, a methanol/propanol mixture, an ethanol/propanol mixture, a methanol/ethanol/propanol mixture, a hydrocarbon naphtha.

In another aspect, the present invention provides a process “B” for producing a hydrogen carrier and transporting it to a site where the hydrogen carrier is converted to hydrogen and carbon dioxide or syngas. The process B involves: producing an H₂ stream; producing or obtaining CO₂ that is converted to a CO₂ stream; catalytically converting the H₂ and CO₂ streams to low carbon syngas; catalytically converting the low carbon syngas to a liquid, low carbon H₂ carrier; transporting the low carbon H₂ carrier, or a portion thereof, to a site; catalytically converting the low carbon H₂ carrier at the site to hydrogen and carbon dioxide or syngas.

The H₂ stream of process B is typically produced from water using an electrolyzer powered by low carbon electricity. In certain cases, the H₂ stream is produced by splitting natural gas into hydrogen and carbon dioxide by steam methane reforming or auto thermal reforming. In other cases, the H₂ stream is produced by pyrolyzing methane using electricity generated heat.

The low carbon electricity referenced in process B can be produced in any suitable way. It can, for instance be produced from: a wind farm, a solar farm, a nuclear power plant, a hydroelectric power plant, a geothermal power plant, or battery storage cells charged with intermittent electricity from wind farms or solar farms.

The carbon dioxide converted to a CO₂ stream in process B can be produced and/or captured in a variety of ways, such as: production of CO₂ by and industrial process; capture of CO₂ from ambient air. Nonlimiting examples of industrial processes include: the combustion of fuels, the oxidation of chemicals, a gasification process, petroleum refining, cement production, fertilizer production, ethanol production, power production, or sewage treatment. Capture of CO₂ from ambient air can involve reaction of CO₂ with one or more amine solvents; chemisorption or physisorption of CO₂ with one or more Metal Organic Framework materials; reaction of CO₂ with a metal oxide material; direct air capture.

In certain cases, the H₂ and CO₂ streams referenced in process B are converted into low carbon syngas in a RWGS reactor. The ration of H₂ to CO₂ fed into the RWGS reactor is between 2.0 ml/mol and 5.0 mol/mol heated to a temperature between 1,450° F. and 1,800° F.

Oftentimes, the low carbon H₂ carrier of process B is methanol or ethanol. The low carbon gas can be converted into methanol, for example, using a Cu—ZnO-based catalyst; the low carbon gas can be converted into ethanol, for example, using three catalysts in tandem reactors. The three catalysts are Cu—Zn-Alkali, Rh—Y-Alkali and Mo—Pd. It can be transported to another site using any suitable means including, without limitation: rail, truck, barge, boat, or pipeline.

In certain cases, the low carbon H₂ carrier of process B is catalytically converted to a mixture containing hydrogen and carbon dioxide using a steam reforming process. The steam reforming process uses a catalyst selected from a Pd—Ag catalyst, a nickel solid solution catalyst or a metal alumina spinel impregnated with one or more Group 1 and Group 2 elements.

In other cases, the low carbon H₂ carrier of process B is catalytically converted to syngas using a catalyst. The catalyst comprises a metal alumina spinel substrate that has a surface area between 50 m²/g and 150 m²/g and that is impregnated with one or more of Cu, Mg, Ni, and Zn at a concentration between 1 part-by-weight and 15 parts-by-weight. The catalyst further includes between 0.1 wt. % and 5 wt. % of La or Ce. The metal alumina spinel substrate is selected from a group of substrates consisting of magnesium aluminate, calcium aluminate, strontium aluminate, potassium aluminate and sodium aluminate. The catalyst typically includes one or two substitutional solid solutions on the metal impregnated metal-alumina spinel. Where the H₂ carrier is methanol, it is oftentimes converted to syngas with a per pass efficiency between 60% and 95% at 100-450 psig; 400-550° F. and a space velocity of 5,000-25,000 hr⁻¹. Where the H₂ carrier is ethanol, it is oftentimes converted to syngas with a per pass efficiency between 45% and 95% at 100-450 psig, 400-550° F. and a space velocity of 5,000-25,000 hr⁻¹.

In certain cases, the H₂ carrier in process B is catalytically converted to syngas, which is then used as an LFP reactor feed. The LFP reactor can be a multi-tubular fixed bed reactor system that is vertically oriented with the LFP reactor feed entering at the top of the LFP reactor.

In another aspect, the present invention provides a catalyst “C” for the conversion of methanol or ethanol to syngas. The catalyst is bound to methanol or ethanol and comprises a metal alumina spinel substrate that has a surface area between 50 m²/g and 150 m²/g. The metal alumina spinel substrate is impregnated with one or two of Cu, Mg, Ni, and Zn at a concentration between 1 part-by-weight and 15 parts-by-weight. The catalyst further includes between 0.1 wt. % and 5 wt. % of La or Ce, and the metal alumina spinel substrate is selected from a group of substrates consisting of magnesium aluminate, calcium aluminate, strontium aluminate, potassium aluminate and sodium aluminate.

In certain cases, the metal alumina spinel substrate of catalyst C is magnesium aluminate or calcium aluminate, and wherein the metal alumina spinel substrate is impregnated with either Cu, Mg or both, and wherein the catalyst further includes La. In other cases, the metal alumina spinel substrate of catalyst C is magnesium aluminate or calcium aluminate, and wherein the metal alumina spinel substrate is impregnated with either Cu, Mg, or both, and wherein the catalyst further includes Ce. In other cases, the metal alumina spinel substrate of catalyst C is strontium aluminate, potassium aluminate or sodium aluminate, and wherein the metal alumina spinel substrate is impregnated with either Cu, Mg, or both, and wherein the catalyst further includes La. In other cases, the metal alumina spinel substrate of catalyst C is strontium aluminate, potassium aluminate or sodium aluminate, and wherein the metal alumina spinel substrate is impregnated with either Cu, Mg, or both, and wherein the catalyst further includes Ce. In other cases, the metal alumina spinel substrate of catalyst C is magnesium aluminate or calcium aluminate, and wherein the metal alumina spinel substrate is impregnated with either Ni, Zn, or both, and wherein the catalyst further includes La. In other cases, the metal alumina spinel substrate of catalyst C is magnesium aluminate or calcium aluminate, and wherein the metal alumina spinel substrate is impregnated with either Ni, Zn, or both, and wherein the catalyst further includes Ce. In other cases, the metal alumina spinel substrate of catalyst C is strontium aluminate, potassium aluminate or sodium aluminate, and wherein the metal alumina spinel substrate is impregnated with either Ni, Zn, or both, and wherein the catalyst further includes La. In other cases, the metal alumina spinel substrate of catalyst C is strontium aluminate, potassium aluminate or sodium aluminate, and wherein the metal alumina spinel substrate is impregnated with either Ni, Zn, or both, and wherein the catalyst further includes Ce. Catalyst C can include one or two substitutional solid solutions on the metal impregnated metal-alumina spinel. Hydrogen can also be bound to the catalyst.

U.S. Patent Application Documents

| | | |
|---|---|---|
| 2003/0113244 A1 | June 2003 | DuPont et al |

U.S. Patent Documents

| | | |
|---|---|---|
| 7,718,832 B1 | May 2010 | Schuetzle et al |
| 8,394,862 B1 | March 2013 | Schuetzle et al |
| 8,741,001 B1 | June 2014 | Schuetzle et al |
| 9,090,831 B2 | July 2015 | Schuetzle et al |
| 9,476,002 B1 | October 2016 | Schuetzle et al |
| 9,611,145 B1 | April 2017 | Schuetzle et al |
| 9,631,147 B1 | April 2017 | Schuetzle et al |
| 10,478,806 B1 | November 2019 | Schuetzle et al |

Foreign Patent Documents

| | | |
|---|---|---|
| GB 1995/2279583 A | November 1995 | Iwanani et al |
| AU 2015/203898 B2 | July 2015 | Landau et al |

### OTHER PUBLICATIONS

Amphlett, J. C., Mann, R. F., Peppley, B. A.: The steam reforming of methanol: mechanism and kinetics compared to the methanol synthesis process: Studies in Surface Science and Catalysis, 81, 409-411 (1994).
Artz, J., Müller, T. E., Thenert, K., Kleinekorte, J., Meys, R., Sternberg, A., Bardow, A, Leitner, W: Sustainable conversion of carbon dioxide: An integrated review of catalysis and life cycle assessment. Chemical Reviews, 118, 434-504 (2018).
Bahmanpour, A. M., Heroguel, F., Kilic, M., Baranowski, C. J., Artiglia, L.: Cu—Al spinel as a highly active and catalyst for the reverse water gas shift reaction. ACS Catal., 9, 6243-6251 (2019).
Carraro, F., Fapohunda, A., Paganini, M. C. Agnoli, S.: Morphology and size effect of ceria nanostructures on the catalytic performances of Pd/CeO₂ catalysts for methanol decomposition to syngas, ACS Appl. Nano Mater, 1 (4) 1492-1501 (2018).
Centi, G., Perathoner, S.: Opportunities and prospects in the chemical recycling of carbon dioxide to fuels. Catalysis Today, 148, 191-205 (2009).
Chen, X., Chen, Y., Song, C., Ji, P., Wang, N., Wang, W., Cui, L.: Recent advances in supported metal catalysts and oxide catalysts for the reverse water-gas shift reaction, Front. Chem., 8, 1-21 (2020)
Choi (2017).
Daza, Y. A., Kuhn, J. N.: CO₂ conversion by reverse water gas shift catalysis: Comparison of catalysts, mechanisms, and their consequences for CO₂ conversion to liquid fuels, Royal Society of Chemistry Advances, 6, 49, 675-49,691 (2016).
Dalena, F., Senatore, A., Basile, M., Knani, S., Basile, A., Iulianelli, A.: Advances in methanol production and utilization, with particular emphasis toward H₂ generation via membrane reactor technology, Membranes, 8, 1-27 (2018)
Fischer, N., Claeys, M., Van Steen, E., Niemantsverdriet, H., Vosloo, M.: Syngas convention—fuels and chemicals from synthesis gas: state of the art, 2, 1-200 (2016).
Gumber, S., Gurumoorthy, A. V. P.: Methanol economy versus H₂ economy, in Methanol: Science and Engineering. Basile, A., Dalena, F., editors. Elsevier; Amsterdam, The Netherlands 661-674 (2018).
Han, J.-T., Xue, Z. H., Zhang, K., Wang, H. H., Li, X. H., Chen, J. S.: Atomically dispersed Ni-based anti-coking catalysts for methanol dehydration in a fixed-bed reactor, ACS Catalysis, 10 (21), 12569-12574 (2020).
Hepburn, C., Adlen, E., Beddington, J., Carter, E. A., Fuss, S., Dowell, N. M., Minx, J. C., Smith, P., Williams, C. K.: The technological and economic prospects for CO₂ utilization and removal, Nature, 575, 87-97 (2019).
Hurskainen, M., Ihonen, J.: Techno-economic feasibility of road transport of H₂ using liquid organic H₂ carriers, International Journal of H₂ Energy, 45 (56), 32098-32112 (2020).
Jiang, Z., Xiao, T., Kuznetsov, V. L., Edwards, P. P.: Turning carbon dioxide into fuel. Phil. Trans. R. Soc. A, 368, 3343-3364 (2010).
Lulianelli, A., Ribeirinha, P., Mendes, A., Basile, A.: Methanol steam reforming for H₂ generation via conventional and membrane reactors: A review, Renewable and Sustainable Energy Reviews, 29, 355-368 (2014).
Li, W., Wang, H., Jiang, X., Zhu, J., Liu, Z., Guo, X., Song, C.: A short review of recent advances in CO₂ hydration to hydrocarbons over heterogeneous catalysts, RSC Adv., 8, 7651 (2018). (2019?)
Lortie, M.: Reverse water gas shift reaction over supported Cu—Ni nanoparticle catalysts, Department of Chemical and Biological Engineering M. S. Thesis, University of Ottawa, Ottawa, Canada (2014).
Matsumurakoji, Y., Tanaka, N., Tode, Tetsuo, Y., Masatake, H.: Catalytic methanol decomposition to carbon monoxide and H₂ over nickel supported on silica, Journal of Molecular Catalysis A: Chemical, 152, 157-165 (2000)
National Academy of Sciences, Chemical Utilization of CO₂ into Chemicals and Fuels, Gaseous Carbon Waste Streams Utilization: Status and Research Needs, National Academies Press, Washington D.C. (2019).
National Energy Technology Laboratory: Syngas conversion to methanol, www.netl.doe.gov) (2021).
Olah, G. A., Goeppert, A., Surya Prakash, G. K.: Chemical recycling of carbon dioxide to methanol and dimethyl ether—from greenhouse gas to renewable, environmentally carbon neutral fuels and synthetic hydrocarbons. J. Org. Chem., 74, 487-498 (2009).
Palo, D. R., Dagle, R. A., Holladay, J. D.: Methanol Steam Reforming for H₂ Production, Chemical Reviews, 107 (10) 399-4021 (2007).
Preuster, P., Papp, C., Wasserscheid, P.: Liquid organic H₂ carriers (LOHC's): toward a H₂-free H₂ economy, Accounts of Chemical Research 50 (1), 74-5 (2017).
Ranjekar, A. M., Yadav, G. D.: Steam reforming of methanol for H₂ production: A critical analysis of catalysis, processes and scope, Ind. Eng. Chem. Res, 60 (1), 89-113 (2021).
Schuetzle, D., Tamblyn, G., Caldwell, M., Schuetzle, R.: Solar reforming of carbon dioxide to produce diesel fuel. U. S. Department of Energy report #DE-FE0002558 (2010).
Schuetzle, D., Tamblyn, G., Caldwell, M., Hanbury, O., Schuetzle, R., Rodriquez, R., Johnson, A., Deichert, F., Jorgensen, R., Struble, D: Demonstration of a pilot integrated biorefinery for the efficient, direct conversion of biomass to diesel fuel. DOE Technical Report #DE-EE0002876, U.S. Department of Energy Bioenergy Technologies Office (DOE-BTO), Golden, CO, 1-261 (May 2015) (www.researchgate.net)
Schuetzle, D.: Historical and predicted global climate changes and some potential accelerated climate moderation approaches, 2018 Global Climate Action Summit, San Francisco, CA, 1-42 (2020) (www.researchgate.net).
Shukla, P. R. et al: Climate Change and Land: an IPCC special report on climate change, desertification, land degradation, sustainable land management, food security, and greenhouse gas fluxes in terrestrial ecosystems, 2019 Intergovernmental Panel on Climate Change (2019) (www.ipcc.ch)
Sun, F-M., Yan, C-F., Wang, Z-D., Guo, C-Q., Huang, S-L.: Ni/Ce on Zr-0 catalyst for high CO₂ conversion during reverse water gas shift reaction (RWGS), International Journal of H₂ Energy, 40 (46), 15985-15993 (2015)
Tan, E. C. D., Schuetzle, D., Zhang, Y., Hanbury, O., Schuetzle, R.: Reduction of greenhouse gas and criteria pollutant emissions by direct conversion of associated flare gas to synthetic fuels at oil wellheads, International Journal of Energy and Environmental Engineering, 9: 305-321 (2018)
Vogt, C., Monai, M., Kramer, G. J., Weckhuysen, B. M.: The renaissance of the Sabatier reaction and its applications on Earth and in space, Nature Catalysis, 2, 188-197 (2019).
Wang, Y., Liu, T., Lei, L., Chen, F.: High temperature solid oxide H₂O/CO₂ co-electrolysis for syngas production, Fuel Processing Technology, 161 (2016).
Williamson, D., Herdes, C., Torrente-Murciano, L., Jones, M., Mattia, D.: N-doped Fe for combined RWGS-FT CO₂ hydration, 7, 7395-7402, ACS Sustainable Chem. Engineering (2019).
Yan, Z., Hitt, J. L., Turner, J. A., Mallouk, T. E.: Renewable electricity storage using electrolysis, Proceedings of the National Academy of Sciences, 117 (23), 12558-12563 (2019).
Xu, X., Shuai, K., Xu, B.: Review on copper and palladium-based catalysts for methanol steam reforming to produce H₂. Catalysts, 7:183 (2017).
Zhu, Q.: Developments on CO₂-utilization technologies, Clean Energy, 3, 85-100 (2019).

1-30. (canceled)

31. A catalyst for the conversion of methanol or ethanol to syngas, wherein the catalyst is bound to methanol or ethanol, and wherein the catalyst comprises a metal alumina spinel substrate that has a surface area between 50 m²/g and 150 m²/g, and wherein the metal alumina spinel substrate is impregnated with one or two of Cu, Mg, Ni, and Zn at a concentration between 1 part-by-weight and 15 parts-by-weight, and wherein the catalyst further includes between 0.1 wt. % and 5 wt. % of La or Ce, and wherein the metal alumina spinel substrate is selected from a group of substrates consisting of magnesium aluminate, calcium aluminate, strontium aluminate, potassium aluminate and sodium aluminate.

32. The catalyst according to claim 31, wherein the metal alumina spinel substrate is magnesium aluminate or calcium aluminate, and wherein the metal alumina spinel substrate is impregnated with either Cu, Mg or both, and wherein the catalyst further includes La.

33. The catalyst according to claim 31, wherein the metal alumina spinel substrate is magnesium aluminate or calcium aluminate, and wherein the metal alumina spinel substrate is impregnated with either Cu, Mg, or both, and wherein the catalyst further includes Ce.

34. The catalyst according to claim 31, wherein the metal alumina spinel substrate is strontium aluminate, potassium aluminate or sodium aluminate, and wherein the metal alumina spinel substrate is impregnated with either Cu, Mg, or both, and wherein the catalyst further includes La.

35. The catalyst according to claim 31, wherein the metal alumina spinel substrate is strontium aluminate, potassium aluminate or sodium aluminate, and wherein the metal alumina spinel substrate is impregnated with either Cu, Mg, or both, and wherein the catalyst further includes Ce.

36. The catalyst according to claim 31, wherein the metal alumina spinel substrate is magnesium aluminate or calcium aluminate, and wherein the metal alumina spinel substrate is impregnated with either Ni, Zn, or both, and wherein the catalyst further includes La.

37. The catalyst according to claim 31, wherein the metal alumina spinel substrate is magnesium aluminate or calcium aluminate, and wherein the metal alumina spinel substrate is impregnated with either Ni, Zn, or both, and wherein the catalyst further includes Ce.

38. The catalyst according to claim 31, wherein the metal alumina spinel substrate is strontium aluminate, potassium aluminate or sodium aluminate, and wherein the metal alumina spinel substrate is impregnated with either Ni, Zn, or both, and wherein the catalyst further includes La.

39. The catalyst according to claim 31, wherein the metal alumina spinel substrate is strontium aluminate, potassium aluminate or sodium aluminate, and wherein the metal alumina spinel substrate is impregnated with either Ni, Zn, or both, and wherein the catalyst further includes Ce.

40. The catalyst according to claim 31, wherein the catalyst includes one or two substitutional solid solutions on the metal impregnated metal-alumina spinel.

41. The catalyst according to claim 31, wherein the catalyst is bound to hydrogen.
